(19) United States
(12) Patent Application Publication
(10) Pub. No.: US 2024/0285903 A1
(43) Pub. Date: Aug. 29, 2024

(54) ATTACHMENT DEVICE FOR A SHEATH HUB HAVING ANGLED SUTURE PADS AND STRAIN RELIEF

(71) Applicants: Boston Scientific Medical Device Limited, Galway (IE); Boston Scientific Scimed, Inc., Maple Grove, MN (US)

(72) Inventors: Neeraj Kumar Sharma, Delhi (IN); Qian Liu, Plymouth, MN (US); Kara Crotta, Albuquerque, NM (US); Elise Moore, Los Angeles, CA (US)

(73) Assignees: Boston Scientific Medical Device Limited, Galway (IE); Boston Scientific Scimed, Inc., Maple Grove, MN (US)

(21) Appl. No.: 18/584,380

(22) Filed: Feb. 22, 2024

Related U.S. Application Data
(60) Provisional application No. 63/447,710, filed on Feb. 23, 2023.

(51) Int. Cl. A61M 25/00; A61M 25/02

(52) U.S. Cl. CPC A61M 25/0097; A61M 2025/0286

(57) ABSTRACT

An attachment device for coupling with a hub includes a base having a proximal end, a distal end, and a lumen extending along a longitudinal axis between the proximal end and the distal end, a first extension coupled with the base, the first extension having a leg portion and a base portion, the base portion comprising at least one opening and a second extension coupled with the base, the second extension having a leg portion and a base portion, the base portion comprising at least one opening. The attachment device includes wherein the leg portion of the first extension and the leg portion of the second portion are each angled relative to the longitudinal axis of the base and wherein the base is composed of an elastomeric material such that the attachment device provides strain relief at both the proximal end and the distal end.

### CROSS-REFERENCE TO RELATED APPLICATIONS

This application claims the benefit of U.S. Provisional Patent Application Ser. No. 63/447,710, filed Feb. 23, 2023, which is incorporated herein by reference.

### TECHNICAL FIELD

The present disclosure relates to attachment means for securing a hub of a sheath to the skin of a patient. More particularly, the present disclosure relates to an attachment device coupled with a hub of an introducer or repositioning sheath, the attachment device having suturing pads for coupling the attachment device with the skin of the patient.

### BACKGROUND

In various procedures for delivering intravascular medical devices, an introducer sheath is inserted percutaneously into a blood vessel of a patient, for example a femoral artery, and medical devices are inserted into the introducer sheath for introduction into the patient's vasculature. In various instances, the medical devices include catheters or other devices such as a blood pump. A hub may be incorporated at a proximal end of the introducer sheath to reduce blood leakage as devices are being inserted, positioned, operated, and removed. The introducer sheath may be removed and replaced by a repositioning sheath, which may also incorporate a hub to maintain hemostasis. During these procedures, it may be desired for the sheath and the hub to be secured to the patient's skin to ensure that the hub and the sheath maintain the desired position. However, during this time, it may also be required that medical devices are delivered through the introducer sheath and/or coupled with the repositioning sheath to change position, or for the sheath and/or hub to change position. There is a need for improved attachments means for securing the hub and related sheath to the patient.

### SUMMARY

In Example 1, an attachment device configured for coupling with a hub of a sheath for delivering a medical device includes a base having a proximal end, a distal end opposite the proximal end, and a lumen extending along a longitudinal axis between the proximal end and the distal end, a first extension coupled with the base, the first extension having a leg portion and a base portion, the base portion comprising at least one opening and a second extension coupled with the base, the second extension having a leg portion and a base portion, the base portion comprising at least one opening. The attachment device further includes wherein the leg portion of the first extension and the leg portion of the second portion are each angled relative to the longitudinal axis of the base and wherein the base is composed of an elastomeric material such that the attachment device is configured to provide strain relief.

In Example 2, the attachment device of Example 1 further includes wherein the elastomeric material is composed of silicone or rubber.

In Example 3, the attachment device of Example 1 or Example 2 further includes wherein the base includes a narrowed portion and a cylindrical portion.

In Example 4, the attachment device of Example 3 further includes wherein the narrowed portion is defined by a length and the cylindrical portion is defined by a length, wherein the length of the narrowed portion is less than the length of the cylindrical portion.

In Example 5, the attachment device of Example 3 further includes wherein the narrowed portion is defined by a length and the cylindrical portion is defined by a length, wherein the length of the narrowed portion is approximately equal to the length of the cylindrical portion.

In Example 6, the attachment device of any of the preceding Examples further includes wherein the angle at which the leg portion of the first extension extends and the angle at which the leg portion of the second extension extends are approximately equal.

In Example 7, the attachment device of Example 6 further includes the angle having a value between approximately 30 degrees and approximately 90 degrees.

In Example 8, the attachment device of any of the preceding Examples further includes wherein at least a portion of the hub is received within the lumen of the attachment device.

In Example 9, the attachment device of any of the preceding Examples further includes wherein the at least one opening of each base portion includes two openings.

In Example 10, a delivery system for at least one medical device into a blood vessel includes a sheath having a proximal end and a distal end and configured for insertion through the blood vessel and a hub engaged with the proximal end of the sheath, the hub coupled with an attachment device at the distal end of the hub. The attachment device includes a base having a proximal end, a distal end opposite the proximal end, and a lumen extending along a longitudinal axis between the proximal end and the distal end, a first extension coupled with the base, the first extension having a leg portion and a base portion, the base portion comprising at least one opening, and a second extension coupled with the base, the second extension having a leg portion and a base portion, the base portion comprising at least one opening. The attachment device further includes wherein the leg portion of the first extension and the leg portion of the second portion are each angled relative to the longitudinal axis of the base and wherein the base is composed of an elastomeric material such that the attachment device is configured to provide strain relief at both the proximal end and the distal end during engagement with the hub.

In Example 11, the delivery system of Example 10 further includes wherein the hub includes a cylindrical portion coupled with a narrowed portion and the cylindrical portion is defined by an outer wall.

In Example 12, the delivery system of Example 11 further includes wherein a wall of the lumen of the base comprises a groove extending circumferentially around the lumen.

In Example 13, the delivery system of Example 12 further includes wherein the outer wall of the cylindrical portion includes a rib extending around a circumference of the outer wall, and wherein the rib is configured for engagement with the groove when the hub is coupled with the attachment device.

In Example 14, the delivery system of any one of Examples 10-13 further includes the elastomeric material of the base being silicone or rubber.

In Example 15, the delivery system of any one of Examples 10-14, wherein the angle at which the leg portion of the first extension extends is between approximately 30 degrees and approximately 90 degrees, and the angle at which the leg portion of the second extension extends is between approximately 30 degrees and approximately 90 degrees.

In Example 16, an attachment device configured for coupling with a hub of a sheath for delivery of a medical device includes a base having a proximal end, a distal end opposite the proximal end, and a lumen extending along a longitudinal axis between the proximal end and the distal end, a first extension coupled with the base, the first extension having a leg portion and a base portion, the base portion comprising at least one opening and a second extension coupled with the base, the second extension having a leg portion and a base portion, the base portion comprising at least one opening. The attachment device further includes wherein the leg portion of the first extension and the leg portion of the second portion are each angled relative to the longitudinal axis of the base and wherein the base is composed of an elastomeric material such that the attachment device is configured to provide strain relief at both the proximal end and the distal end during engagement with the hub.

In Example 17, the attachment device of Example 16 further includes wherein the elastomeric material is composed of silicone or rubber.

In Example 18, the attachment device of Example 16 or Example 17 further includes wherein the base includes a narrowed portion and a cylindrical portion.

In Example 19, the attachment device of Example 18 further includes wherein the narrowed portion is defined by a length and the cylindrical portion is defined by a length, wherein the length of the narrowed portion is less than the length of the cylindrical portion.

In Example 20, the attachment device of Example 18 further includes wherein the narrowed portion is defined by a length and the cylindrical portion is defined by a length, wherein the length of the narrowed portion is approximately equal to the length of the cylindrical portion.

In Example 21, the attachment device of Example 16 further includes wherein the angle at which the leg portion of the first extension extends and the angle at which the leg portion of the second extension are approximately equal.

In Example 22, the attachment device of Example 21 further includes the angle having a value between approximately 30 degrees and approximately 90 degrees.

In Example 23, the attachment device of Example 16 further includes wherein at least a portion of the hub is received within the lumen of the attachment device.

In Example 24, the attachment device of Example 16 further includes wherein the at least one opening of each base portion includes two openings.

In Example 25, an attachment device configured for coupling with a hub of a sheath for delivery of a medical device includes a base having a proximal end, a distal end opposite the proximal end, and a lumen extending along a longitudinal axis between the proximal end and the distal end, the base having a cylindrical portion and a narrowed portion, a first suture pad coupled with the base having at least one opening, and a second suture pad coupled with the base having at least one opening. The attachment device further includes wherein each of the first suture pad and the second suture pad are angled relative to the longitudinal axis of the base with an angle of between approximately 30 degrees and approximately 90 degrees and wherein the cylindrical portion has a length of approximately 10 mm and the narrowed portion has a length of approximately 12 mm.

In Example 26, the attachment device of Example 25 further includes wherein the base is formed of an elastomeric material such that the attachment device is configured to provide strain relief at both the proximal end and the distal end during engagement with the hub.

In Example 27, the attachment device of Example 26 further includes wherein the elastomeric material is silicone or rubber.

In Example 28, the attachment device of Example 25 further includes wherein the first suture pad and the second suture pad each includes at least two openings.

In Example 29, the attachment device of Example 25 further includes wherein the first suture pad and the second suture pad each includes a leg portion extending from the base and attached to a base portion of the first suture pad and the second suture pad.

In Example 30, a delivery system for at least one medical device into a blood vessel includes a sheath having a proximal end and a distal end and configured for insertion through the blood vessel and a hemostasis valve hub engaged with the proximal end of the sheath, the hub coupled with an attachment device at the distal end of the hemostasis valve hub. The attachment device includes a base having a proximal end, a distal end opposite the proximal end, and a lumen extending along a longitudinal axis between the proximal end and the distal end, a first extension coupled with the base, the first extension having a leg portion and a base portion, the base portion comprising at least one opening, and a second extension coupled with the base, the second extension having a leg portion and a base portion, the base portion comprising at least one opening. The attachment device further includes wherein the leg portion of the first extension and the leg portion of the second portion are each angled relative to the longitudinal axis of the base and wherein the base is composed of an elastomeric material such that the attachment device is configured to provide strain relief at both the proximal end and the distal end during engagement with the hemostasis valve hub.

In Example 31, the delivery system of Example 30 further includes wherein the hemostasis valve hub includes a cylindrical portion coupled with a narrowed portion and the cylindrical portion is defined by an outer wall.

In Example 32, the delivery system of Example 31 further includes wherein a wall of the lumen of the base comprises a groove extending circumferentially around the lumen.

In Example 33, the delivery system of Example 32 further includes wherein the outer wall of the cylindrical portion includes a rib extending around a circumference of the outer wall, and wherein the rib is configured for engagement with the groove when the hemostasis valve hub is coupled with the attachment device.

In Example 34, the delivery system of Example 30 further includes the elastomeric material of the base being silicone or rubber.

In Example 35, the delivery system of Example 30, wherein the angle at which the leg portion of the first extension extends is between approximately 30 degrees and approximately 90 degrees, and the angle at which the leg portion of the second extension extends is between approximately 30 degrees and approximately 90 degrees.

### BRIEF DESCRIPTION OF THE DRAWINGS

FIG. 1 illustrates a side view of an introducer sheath extending into a blood vessel, in accordance with elements of the present disclosure.

FIG. 2 illustrates a cross-sectional view of a medical device positioned within a blood vessel, in accordance with embodiments of the present disclosure.

FIG. 3 illustrates a side view of a portion of the introducer sheath having an attachment device and a hub attached to the introducer sheath, in accordance with embodiments of the present disclosure.

FIG. 4 illustrates a top left side perspective view of an attachment device, in accordance with embodiments of the present disclosure.

FIG. 5 illustrates a cross-sectional view of the attachment device of FIG. 4.

FIG. 6 illustrates a top left side perspective view of an attachment device, in accordance with embodiments of the present disclosure.

FIG. 7 illustrates a cross-sectional view of the attachment device of FIG. 6.

FIG. 8 is an exploded view of an attachment device and a hub, in accordance with embodiments of the present disclosure.

FIG. 9 is a cross-sectional view of an attachment device coupled with a hub, in accordance with embodiments of the present disclosure.

### DETAILED DESCRIPTION

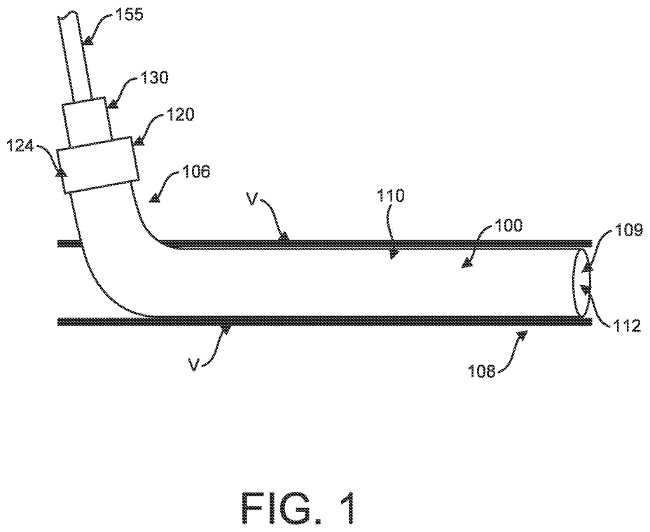

FIG. 1 illustrates a side cross-sectional view of a blood vessel V with an introducer sheath 100, inserted at least partially into the blood vessel V. While the disclosure herein is made with reference largely to the introducer sheath 100, the disclosure may also apply to a repositioning sheath or various other applicable types of sheaths. In some embodiments, the introducer sheath 100 is used for facilitating the passage of various relatively large medical devices, such as a blood pump as will be described further herein, through the introducer sheath 100 and into the blood vessel V. Hence, the introducer sheath 100 may be referred to as a large bore introducer sheath. The introducer sheath 100 comprises a proximal end 106 and a distal end 108 that is opposite the proximal end 106. The introducer sheath 100 includes a proximal opening (not shown) adjacent the proximal end 106 and a distal opening 109 adjacent the distal end 108. A body portion 110 of the introducer sheath 100 extends between the proximal end 106 and the distal end 108, and the body portion 110 defines a lumen 112 of the introducer sheath 100. The introducer sheath 100 may be formed by various polymeric or metallic materials. In further embodiments, the introducer sheath 100 may comprise an additional surface coating. The surface coating may include, but is not limited to, silicone, PET, or any other applicable polymer.

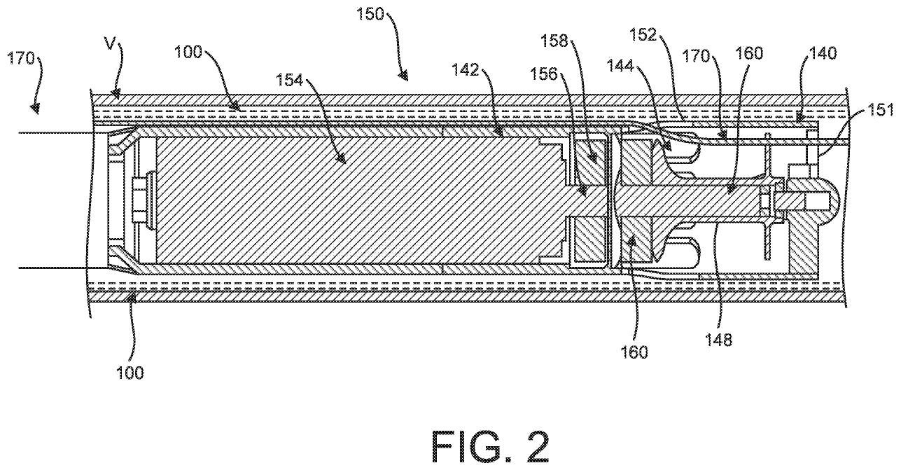

A hub 120 is commonly included at the proximal end 106 and over the proximal opening (not shown) of the introducer sheath 100. The hub 120, also referred to herein as a hemostasis valve hub, is configured for hemostasis, i.e., to prevent blood from leaking out of the introducer sheath 100 during use. More specifically, a medical device, for example a catheter 155, may be inserted through the hub 120 and the introducer sheath 100 and into the blood vessel V, and the hub 120 may maintain hemostasis between the catheter 155, the introducer sheath 100, and the external surroundings. In some embodiments, the catheter 155 may couple to a medical device, as the blood pump 150 shown in FIG. 2. After insertion of the catheter 155, fixation of the axial and radial position of the catheter 155 may be desired to ensure that the catheter 155 (and any coupled medical device) is in the proper position during use. Further, in some instances, it may also be desired for the operator to reposition the catheter 155 (and any coupled medical device) after insertion. As such, the hub 120 may comprise an inbuilt tightening port 130 composed of several components coupled to the hub 120 that provides for the fixation of the catheter 155 with respect to the hub 120 and blood vessel V. It may also be desired to secure the hub 120 to the patient, as described in more detail below.

FIG. 2 illustrates a cross-sectional view of the introducer sheath 100 of FIG. 1 after insertion of a medical device, illustratively a blood pump 150, into the introducer sheath 100. As noted above, in some embodiments a catheter, such as the catheter 155, may be coupled to the proximal end of the blood pump 150 and extend outside the blood vessel V and introducer sheath 100. The blood pump 150 generally includes an impeller assembly housing 140 and a motor housing 142. In some embodiments, the impeller assembly housing 140 and the motor housing 142 may be integrally or monolithically constructed. The impeller assembly housing 140 carries an impeller assembly 144 therein. The impeller assembly 144 includes an impeller shaft 146 and an impeller 148 that rotates relative to the impeller assembly housing 140 to drive blood through the blood pump 150. More specifically, the impeller 148 causes blood to flow from a blood inlet 151 formed on the impeller assembly housing 140, through the impeller assembly housing 140, and out of a blood outlet 152 formed on the impeller assembly housing 140. In some embodiments the impeller shaft 146 and the impeller 148 may be integrated, and in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may couple to a distally extending cannula (not shown), and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components. While the introducer sheath 100 is illustrated above with the use of the blood pump 150, various other medical devices may be used in conjunction with the introducer sheath 100 and the hemostasis valve hub 120.

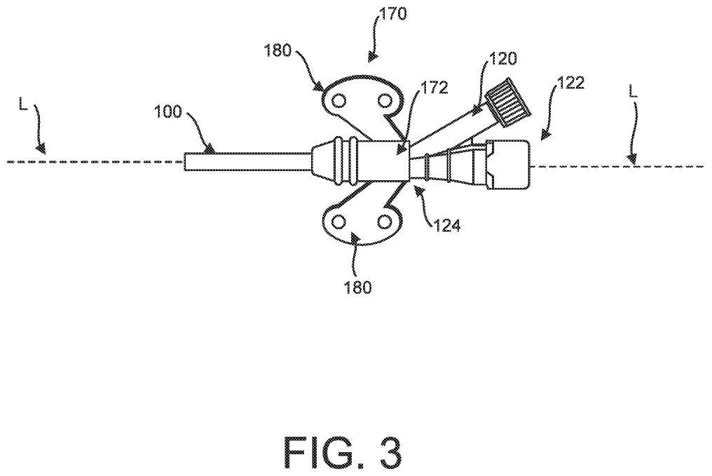

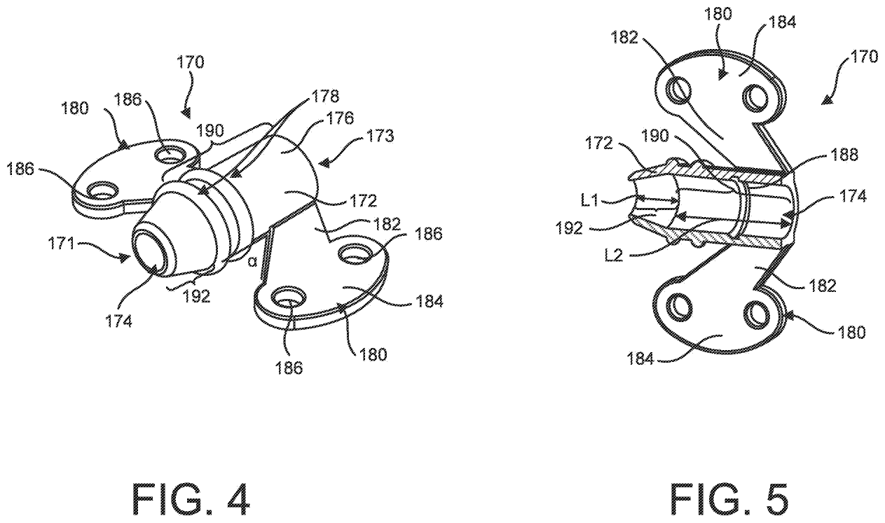

As noted above, it may be desired to secure the hemostasis valve hub 120 against the patient once the introducer sheath 100 or repositioning sheath is introduced into the blood vessel V. For example, as illustrated in the FIG. 3, the hub 120 may have an attachment device 170 coupled with the hub 120. More particularly, the attachment device 170 may be coupled with a distal end 124 of the hub 120. With reference now to FIGS. 4-7, embodiments of the attachment device 170 will be described further herein.

FIG. 4 illustrates a top left side perspective view of the attachment device 170. The attachment device 170 includes a base 172 defined by a generally cylindrical configuration. The base 172 may have a distal end 171 opposite a proximal end 173 and a lumen 174 extending therebetween. The lumen 174 extends along a longitudinal axis L (FIG. 3) of the base 172 of the attachment device 170. Further, the base 172 includes an outer surface 176 having at least two collars 178 extending around the entire circumference of the outer surface 176 of the base 172. As illustrated, the attachment device 170 may also include at least two extensions, or suture pads, 180 extending outwardly from the base 172. As illustrated, each of the at least two suture pads 180 is composed of a connector, or leg portion, 182 and a suture pad base 184. More particularly, the connector 182 extends from the base 172 to the suture pad base 184. As illustrated, each suture pad base 184 is generally oval in shape having at least one opening 186 extending therethrough. Other shapes of the suture pad base 184 are also possible. Once the introducer sheath 100 is inserted into the blood vessel V, the at least one opening 186 may be configured for receiving sutures therethrough to secure the at least two suture pads 180 against the skin of the patient.

Further, with reference still to FIG. 4, the suture pads 180, and more particularly, the connectors, or leg portions, 182 of each suture pad 180 are angled relative to the longitudinal axis L of the base 172 by an angle α. In these embodiments, it is assumed that outer surface 176 of base 172 extends generally parallel with longitudinal axis L and thus, angle α may also be relative to outer surface 176. However, in some embodiments, outer surface 176 of base 172 is not parallel with longitudinal axis L and in these instances, angle α is not defined relative to outer surface 176. The angle α has a value that may range from approximately 30 degrees to approximately 90 degrees. For example, in some embodiments, the angle α has a value that may range from approximately 35 degrees to approximately 85 degrees, from approximately 40 degrees to approximately 80 degrees, from approximately 45 degrees to approximately 75 degrees, from approximately 50 degrees to approximately 70 degrees or from approximately 55 degrees to approximately 65 degrees. In further embodiments, the angle α may have a value of approximately 45 degrees. In this way, each of the at least two suture pads 180 are angled toward the distal end 171 of the base 172 of the attachment device 170.

The forward angulation of each of the at least two suture pads 180 relative to the longitudinal axis L provides several advantages. For example, the forward angulation of the leg portions 182 of each suture pad 180 of the attachment device 170 increases the case with which the operator may adjust the hub 120 before, during or after the procedure, as compared to suture pads without angled connectors. In particular, the operator may more easily actuate the hub 120 either in an upward or a downward direction, which allows for easier insertion of devices into the hub 120. The forward angulation of the leg portions 182 allows the operator to more easily adjust the hub 120 relative to the skin surface, as compared to suture pads without angled connectors, because the openings 186 are positioned closer to the distal end 171 of the attachment device 170 than suture pads without angulation. By moving the openings 186, and hence the connection points between the attachment device 170 and the patient, more distally (as compared to non-angulated suture pads), the proximal end 173 of the attachment device 170 may be more easily lifted from the patient's skin surface. In other words, the hub 120 coupled to the attachment device 170, may be more easily “tented.” In turn, less force is imparted to the leg portions 182 during the adjustment of the hub 120, reducing patient discomfort. In addition, the forward angulation of suture pads 180 helps avoid forward tension migration of the introducer sheath 100 due to ability to more easily adjust and/or “tent” the hub 120. If the leg portions 182 did not have this forward angulation, when adjustment of the hub 120 is required, the operator would likely need to remove the connection of the openings 186 to the patient (e.g., sutures), adjust the hub 120, and resecure leg portions 182 to the patient. Thus, the forward angulation of the leg portions 182 increases the flexibility with which the hub 120 can be adjusted while maintaining connection with the patient, reduces discomfort to the patient, reduces the likelihood of re-suturing of the suture pad, and helps to decrease the forces imparted to the introducer sheath 100 when delivering devices through the hub 120.

The base 172 may be defined as having a cylindrical portion 190 and a narrowed portion 192. The narrowed portion 192 may comprise the distal end 171 of the base 172 while the cylindrical portion 190 may comprise the proximal end 173 of the base 172. As illustrated in the cross-sectional view of FIG. 5, the narrowed portion 192 may have a length L1 and the cylindrical portion 190 may have a length L2. In the illustrative embodiment of FIG. 5, the length L1 may have a value of approximately 6 mm to approximately 8 mm and the length L2 may have a value of approximately 8 mm to approximately 11 mm. In these instances, the length L1 may be less than the length L2. However, in other embodiments, the length L2 may be approximately equal to and/or greater than the length L1.

Further, the base 172, including both the narrowed portion 192 and the cylindrical portion 190 of the base 172, provide a strain relief function. More specifically, the base 172 may be composed of a flexible or compliant material. For example, the base 172 may be composed of an elastomer, including but not limited to, a silicone, rubber, thermoplastic vulcanizate, or various other applicable materials. Once the distal end 124 of the hub 120 is inserted into the lumen 174 of the attachment device 170, at least a majority of the cylindrical portion 190 receives a portion of the hub 120 inserted therethrough. As illustrated in FIG. 3, the narrowed portion 192 receives at least a portion of the introducer sheath 100 extending therethrough. In this way, during operation of the introducer sheath 100 and the hub 120, if there is force imparted onto the hub 120 due to pushing the hub 120 into the attachment device 170, rotating or angling the hub 120, or otherwise moving the hub and/or the introducer sheath 100, the force is imparted onto the elastomeric material of the attachment device 170 rather than the rigid material of the hub 120. In this way, the incorporation of the attachment device 170 provides strain relief that may protect the longevity and/or integrity of the hub 120. Further, as the introducer sheath 100 extends out of the distal end 171 of the attachment device 170, if there is any force imparted onto the introducer sheath 100 toward the distal end 171 of the attachment device 170, the strain and/or force is imparted onto the elastomeric material of the attachment device 170, rather than the introducer sheath 100. This may reduce the chance of the introducer sheath 100 kinking or breaking. In some embodiments, the strain and/or force is imparted on the attachment device 170 rather than the skin of the patient, reducing damage to the patient at an access site within the skin.

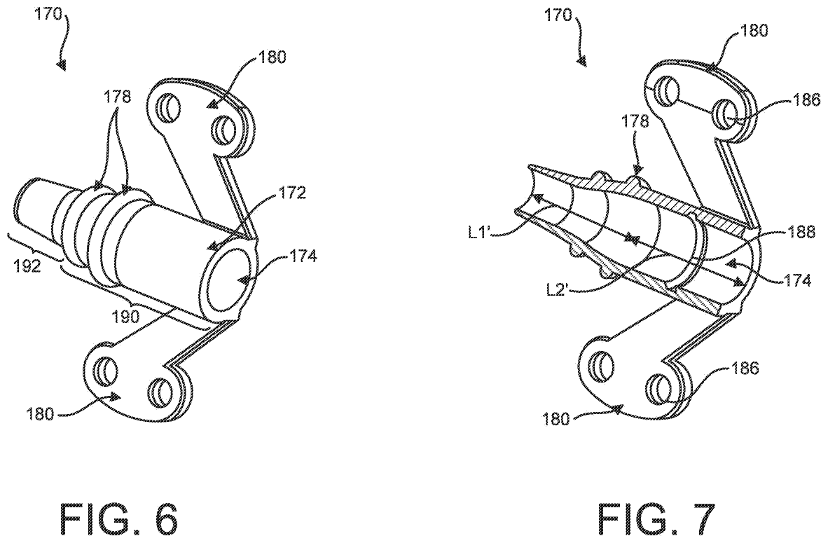

With reference now to FIGS. 6 and 7, an additional embodiment of the attachment device 170 is illustrated. As shown, the attachment device 170 includes the base 172 and the at least two suture pads 180 extending outwardly from the base 172. The base 172 includes the cylindrical portion 190 and the narrowed portion 192. The cylindrical portion 190 is defined by a modified length L2′ and the narrowed portion 192 has a modified length L1′. In the illustrative embodiment of FIGS. 6 and 7, the narrowed portion 192 has a greater length L1′ than the length L1 shown in the illustrative embodiment of FIGS. 4 and 5. In some embodiments, the length L1′ of the narrowed portion 192 may be equal to or greater than the length L2′ of the cylindrical portion 190. In some embodiments, the value of the length L1′ may range between approximately 12 mm to approximately 16 mm and the value of the length L2′ may range between approximately 10 mm and approximately 12 mm. This may provide several advantages when the introducer sheath 100 is inserted into the blood vessel V and the attachment device 170 is pressed against the skin of the patient. For example, this may increase the total volume of the elastomeric material of the attachment device 170 that is capable for providing strain relief. Additionally, the increased length of the narrowed portion 192 increases the depth at which the attachment device 170 can be extended into the patient's skin. This may increase the stability of the connection between the attachment device 170 and the patient and reduce the chance of dislodging or removal of the attachment means 170 from the incision site of the patient. Further, the increased length of the narrowed portion 192, as indicated by length L1′, may additionally increase the ability of the hub 120 to seal the insertion site for the introducer sheath 100, and thus decrease the amount of blood leakage and loss around the introducer sheath 100 and the hub 120. More particularly, the increased length L1′ allows for more of the incision site to be occupied by the attachment device 170, reducing the ability of blood to seep around the attachment device 170 and out of the incision site.

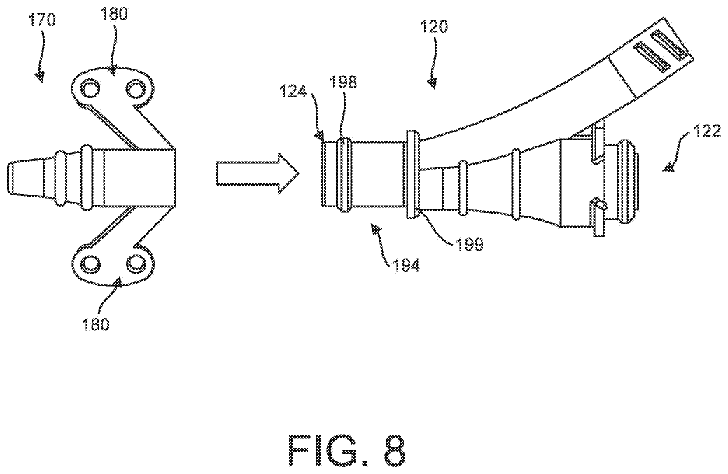

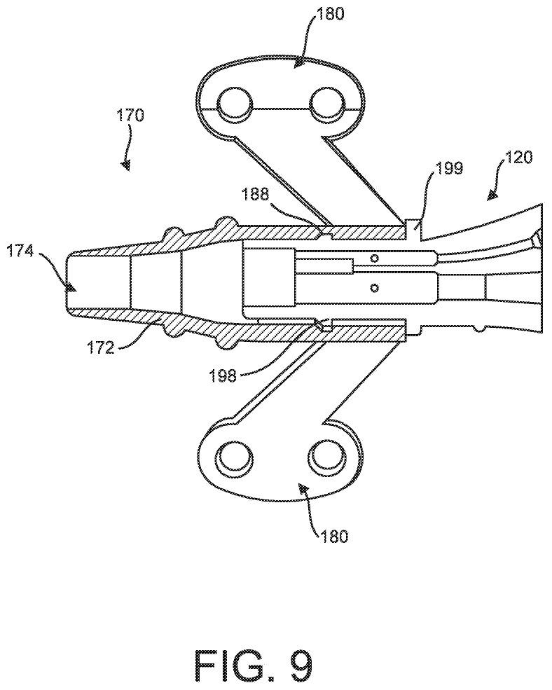

FIG. 8 illustrates an exploded view of the hub 120 and the attachment device 170. As illustrated, the hub 120 includes a proximal end 122 opposite the distal end 124. The distal end 124 is defined by an engagement portion 194 which is configured to be received within the lumen 174 of the attachment device 170. With reference still to FIG. 8, the engagement portion 194 is defined as having an outer wall 196 which has a generally cylindrical shape. The outer wall 196 includes at least one engagement rib 198 extending therefrom. In these embodiments, the engagement rib 198 is configured for reception with a groove 188 of lumen 174 of the attachment device 170, shown, for example in FIGS. 5 and 7. The outer wall 196 additionally includes a stop ring 199 which extends from the outer wall 196 and extends circumferentially around the outer wall 196. As illustrated in FIG. 9, the stop ring 199 acts as an axial stop when the hub 120 is pushed into the lumen 174 of the attachment device 170 to indicate to the user when the hub 120 is entirely inserted into the attachment device 170 (i.e., the groove 188 is engaged with the engagement rib 198).

In this way, the attachment device 170 may be pushed onto the distal end 124 of the hub 120 until the groove 188 is engaged with the engagement rib 198 to secure the positioning of the attachment device 170 onto the hub 120. FIG. 9 illustrates a cross-sectional view of the hub 120 with the attachment device 170 fully engaged and placed onto the hub 120. More particularly, the engagement rib 198 of the hub 120 is engaged within the groove 188 of the hub 120. In this way, the hub 120 and the attachment device 170 are still capable of rotation relative to one another as the engagement between the hub 120 and the attachment device 170 is retained.

While the embodiments and method are described with reference to the catheter 155 and the blood pump 150 for reception into the catheter 155, various other medical devices may be incorporated. Various modifications and additions can be made to the exemplary embodiments discussed without departing from the scope of the present invention. For example, while the embodiments described above refer to particular features, the scope of this invention also includes embodiments having different combinations of features and embodiments that do not include all of the above-described features.

What is claimed is:

1. An attachment device configured for coupling with a hub of a sheath for delivery of a medical device, the attachment device comprising:
a base having a proximal end, a distal end opposite the proximal end, and a lumen extending along a longitudinal axis between the proximal end and the distal end;
a first extension coupled with the base, the first extension having a leg portion and a base portion, the base portion comprising at least one opening;
a second extension coupled with the base, the second extension having a leg portion and a base portion, the base portion comprising at least one opening;
wherein the leg portion of the first extension and the leg portion of the second portion are each angled relative to the longitudinal axis of the base; and
wherein the base is composed of an elastomeric material such that the attachment device is configured to provide strain relief at both the proximal end and the distal end when the attachment device is coupled with the hub.

2. The attachment device of claim 1, wherein the elastomeric material is composed of silicone or rubber.

3. The attachment device of claim 1, wherein the base comprises a narrowed portion and a cylindrical portion.

4. The attachment device of claim 3, wherein the narrowed portion is defined by a length and the cylindrical portion is defined by a length, wherein the length of the narrowed portion is less than the length of the cylindrical portion.

5. The attachment device of claim 3, wherein the narrowed portion is defined by a length and the cylindrical portion is defined by a length, wherein the length of the narrowed portion is approximately equal to the length of the cylindrical portion.

6. The attachment device of claim 1, wherein the angle at which the leg portion of the first extension extends and the angle at which the leg portion of the second extension are approximately equal.

7. The attachment device of claim 7, wherein the angle has a value between approximately 30 and approximately 90.

8. The attachment device of claim 1, wherein at least a portion of the hub is received within the lumen of the attachment device.

9. The attachment device of claim 1, wherein the at least one opening of each base portion includes two openings.

10. An attachment device configured for coupling with a hub of a sheath for delivery of a medical device, the attachment device comprising:
a base having a proximal end, a distal end opposite the proximal end, and a lumen extending along a longitudinal axis between the proximal end and the distal end, the base having a cylindrical portion and a narrowed portion;
a first suture pad coupled with the base having at least one opening;
a second suture pad coupled with the base having at least one opening;
wherein each of the first suture pad and the second suture pad are angled relative to the longitudinal axis of the base with an angle of between approximately 30 degrees and approximately 90 degrees; and
wherein the cylindrical portion has a length of approximately 10 mm and the narrowed portion has a length of approximately 12 mm.

11. The attachment device of claim 10, wherein the base is formed of an elastomeric material such that the attachment device is configured to provide strain relief at both the proximal end and the distal end when coupled with the hub.

12. The attachment device of claim 11, wherein the elastomeric material is silicone or rubber.

13. The attachment device of claim 10, wherein the first suture pad and the second suture pad each includes at least two openings.

14. The attachment device of claim 10, wherein the first suture pad and the second suture pad each includes a leg portion extending from the base and attached to a base portion of the first suture pad and the second suture pad.

15. A delivery system for at least one medical device into a blood vessel, the delivery system comprising:
a sheath having a proximal end and a distal end and configured for insertion through the blood vessel;
a hemostasis valve hub coupled with the proximal end of the sheath, the hemostasis valve coupled with an attachment device at the distal end of the hemostasis valve hub, the attachment device comprising:
a base having a proximal end, a distal end opposite the proximal end, and a lumen extending along a longitudinal axis between the proximal end and the distal end;
a first extension coupled with the base, the first extension having a leg portion and a base portion, the base portion comprising at least one opening;
a second extension coupled with the base, the second extension having a leg portion and a base portion, the base portion comprising at least one opening;
wherein the leg portion of the first extension and the leg portion of the second portion are each angled relative to the longitudinal axis of the base; and
wherein the base is composed of an elastomeric material such that the attachment device is configured to provide strain relief at both the proximal end and the distal end when coupled with the hemostasis valve hub.

16. The delivery system of claim 15, wherein the hemostasis valve hub includes a cylindrical portion coupled with a narrowed portion and the cylindrical portion is defined by an outer wall.

17. The delivery system of claim 16, wherein a wall of the lumen of the base comprises a groove extending circumferentially around the lumen.

18. The delivery system of claim 17, wherein the outer wall of the cylindrical portion comprises a rib extending around a circumference of the outer wall, and wherein the rib is configured for engagement with the groove when the hemostasis valve hub is coupled with the attachment device.

19. The delivery system of claim 15, wherein the elastomeric material of the base is silicone or rubber.

20. The delivery system of claim 15, wherein the angle at which the leg portion of the first extension extends is between approximately 30 degrees and approximately 90 degrees, and the angle at which the leg portion of the second extension extends is between approximately 30 degrees and approximately 90 degrees.
